(19) United States
(12) Patent Application Publication
(10) Pub. No.: US 2008/0194626 A1
(43) Pub. Date: Aug. 14, 2008

(54) ANTIHYPERTENSIVE DRUG COMBINATION

(76) Inventor: Richard J. Gorczynski, Westminster, CO (US)

Correspondence Address: HARNESS, DICKEY, & PIERCE, P.L.C, 7700 Bonhomme, Suite 400, ST. LOUIS, MO, 63105, US

(21) Appl. No.: 12/028,079

(22) Filed: Feb. 8, 2008

Related U.S. Application Data
(60) Provisional application No. 60/889,873, filed on Feb. 14, 2007.

(51) Int. Cl. A61K 31/445; A61P 9/12

(52) U.S. Cl. 514/317

(57) ABSTRACT

Therapeutic combinations and pharmaceutical compositions are provided comprising darusentan and an inhibitor of renin activity or release in absolute and relative amounts effective to provide a beneficial change in a subject's 24-hour pattern of systolic and/or diastolic blood pressure. There are further provided methods of using such combinations or compositions to treat hypertensive disorders, or to lower blood pressure in subjects exhibiting resistance to a baseline antihypertensive therapy.

This application claims the benefit of U.S. provisional application Ser. No. 60/889,873, filed Feb. 14, 2007, incorporated in its entirety herein by reference.

### FIELD OF THE INVENTION

The present invention relates to therapeutic combinations, compositions and methods useful for management and control of blood pressure.

### BACKGROUND OF THE INVENTION

Hypertension continues to have a large prevalence in society. An estimated 50 million people suffer from hypertension in the United States alone. According to the Seventh Report of the Joint National Committee on Prevention, Detection, Evaluation, and Treatment of High Blood Pressure (JNC 7; Chobanian et al. (2003) Hypertension 42 1206-1252), a goal of systolic blood pressure (SBP)<140 mmHg and diastolic blood pressure (DBP)<90 mmHg is recommended for patients with hypertension and no other serious conditions.

Antihypertensive therapies involving drugs having various modes of action are well established but are not always effective and not always well tolerated by patients. Effectiveness of an antihypertensive drug therapy can be monitored by occasional measurement of blood pressure, for example during a visit to a clinic or physician's office, but such monitoring does not give a full picture of the patient's response to the therapy and can lead to missed opportunities to provide better management or control of blood pressure. For example, there are particular times of the day that blood pressure tends to be elevated and at such times it is especially important that a therapy is effective in lowering blood pressure. In addition, particularly for patients on a once daily drug administration regimen, the “trough” period when the concentration of the drug in the bloodstream is at its lowest can be a particularly vulnerable time. Thus, an important and often underappreciated aspect of blood pressure control is providing a beneficial change in the patient's 24-hour pattern of SBP and/or DBP.

For patients with serious or compelling conditions such as diabetes and chronic kidney disease, JNC 7 recommends a goal of SBP<130 mmHg and DBP<80 mmHg. It should be noted that the British Hypertensive Society (BHD-IV; J. Human Hypertens. (2004) 18:139-185), the European Society of Hypertension/European Society of Cardiology (ESH/ESC; J. Hypertens. (2003) 21:1011-1053), and the World Health Organization/International Society of Hypertension (WHO/ISH; J. Hypertens. (2003) 21:1983-1992) guidelines propose blood pressure goals for non-diabetic and diabetic patients that are similar but not identical to the JNC 7 guidelines. Despite intensive, multi-drug therapy, only about 50% of patients with diabetes or chronic kidney disease reach traditional blood pressure goals, with even fewer reaching the more stringent goals now recommended by JNC 7. Thus, patients with serious conditions such as diabetes and/or chronic kidney disease represent a particularly challenging population for antihypertensive drug therapy.

Those resistant to a baseline antihypertensive therapy also represent a challenging patient population. Resistant hypertension is defined by JNC 7 as a failure to achieve goal blood pressure in patients who are adhering to full doses of an appropriate three-drug regimen that includes a diuretic. In particular, those patients having both resistant hypertension and a serious condition such as diabetes and/or chronic kidney disease represent an especially challenging population for antihypertensive drug therapy.

Darusentan is an endothelin-A (ETA) selective receptor antagonist which has been used to treat moderate hypertension. Endothelin (more particularly the ET-1 isoform thereof) is a small peptide hormone that is believed to play a critical role in control of blood flow and cell growth. Elevated endothelin blood levels are associated with several cardiovascular disease conditions, including pulmonary arterial hypertension, chronic renal disease, coronary artery disease, hypertension, and chronic heart failure. Endothelin is a potent vasoconstrictor, triggering contraction through endothelin-receptor mediated signaling pathways. While antagonism of the ETA receptor is known to reduce endothelin-mediated vasoconstriction, antagonism of the endothelin-B (ETB) receptor can block clearance of ET-1 from the circulatory system, exacerbating its hypertensive effect.

Renin is an aspartyl protease secreted by the kidneys. Its primary substrate is angiotensinogen, which is secreted by hepatocytes. Renin cleaves angiotensinogen forming the decapeptide angiotensin I. Angiotensin I is then further cleaved in the lungs to release the octapeptide, angiotensin II. Angiotensin II increases blood pressure both directly by arterial vasoconstriction and indirectly by promoting the removal of aldosterone, a sodium-ion retaining hormone, from the adrenal glands, thus increasing extracellular fluid volume. Renin inhibitors can affect either the activity or release of renin from the kidney. By either affecting the activity or release of renin, less angiotensin I is formed from the cleavage of angiotensinogen. This results in less angiotensin II production and a reduction in blood pressure.

International Patent Publication No. WO 02/40007 of Novartis proposes combinations of the renin inhibitor aliskiren and any of a large number of therapeutic agents of many different classes for use in treating cardiovascular diseases such as hypertension and atherosclerosis. One such class mentioned is endothelin antagonists, of which darusentan is named as one of several examples.

International Patent Publication No. WO 03/099767 of Novartis proposes certain amide derivatives as inhibitors of the enzymatic activity of renin, and combinations of such amide derivatives with any of a large number of therapeutic agents of many different classes for use in treating various disorders such as hypertension and congestive heart failure. One such class mentioned is endothelin antagonists, of which darusentan is named as one of several examples.

International Patent Publication No. WO 04/100871 of Pharmacia proposes inter alia therapeutic combinations and compositions comprising an aldosterone receptor antagonist, a renin inhibitor and a third drug, which can be any of a large number of drugs of different classes. One such class mentioned is endothelin receptor antagonists, of which darusentan is named as one of several examples.

Despite current therapies, there is still a need to control blood pressure in the patient population. In particular, improved drug therapies for hypertensive disorders that can provide a beneficial change in a patient's 24-hour pattern of SBP and/or DBP are much needed in the art. Furthermore, new methods to lower blood pressure in patients exhibiting resistance to a baseline antihypertensive therapy, to prevent cardiovascular adverse events in such patients and to provide beneficial effects on renal function in such patients would be highly desirable.

### SUMMARY OF THE INVENTION

There is now provided a method for treating a hypertensive disorder in a subject, comprising administering to the subject in combination therapy darusentan and an inhibitor of renin activity or release, in absolute and relative amounts effective to provide a beneficial change in the subject's 24-hour pattern of systolic and/or diastolic blood pressure.

There is further provided a method for treating a hypertensive disorder in a subject. The method comprises administering to the subject in combination therapy darusentan and an inhibitor of renin activity or release, in absolute and relative amounts effective to provide a beneficial change in the subject's 24-hour pattern of systolic and/or diastolic blood pressure.

There is still further provided a method for lowering blood pressure in a subject exhibiting resistance to a baseline antihypertensive therapy with one or more drugs. The method comprises administering to the subject in combination therapy darusentan and an inhibitor of renin activity or release.

There is still further provided a method for preventing one or more cardiovascular adverse events in a subject having resistant hypertension. The method comprises administering to the subject in combination therapy darusentan and an inhibitor of renin activity or release.

There is still further provided a method for providing a beneficial effect on renal function in a subject having resistant hypertension. The method comprises administering to the subject in combination therapy darusentan and an inhibitor of renin activity or release.

There is still further provided a therapeutic combination comprising darusentan and an inhibitor of renin activity or release in absolute and relative amounts effective (a) to provide a beneficial change in a subject's 24-hour pattern of SBP and/or DBP; and/or (b) to lower blood pressure in a subject exhibiting resistance to a baseline antihypertensive therapy with one or more drugs; wherein the darusentan and the inhibitor of renin activity or release are each formulated for once-daily oral administration.

There is further provided a pharmaceutical composition comprising darusentan, an inhibitor of renin activity or release, and at least one pharmaceutically acceptable excipient; wherein the darusentan and the inhibitor of renin activity or release are present in absolute and relative amounts effective (a) to provide a beneficial change in a subject's 24-hour pattern of SBP and/or DBP; and/or (b) to lower blood pressure in a subject exhibiting resistance to a baseline antihypertensive therapy with one or more drugs; and wherein the composition is formulated for once-daily oral administration.

Other embodiments, including particular aspects of the embodiments summarized above, will be evident from the detailed description that follows.

### DETAILED DESCRIPTION

In various aspects of the invention, therapeutic combinations and compositions comprising darusentan and an inhibitor of renin activity or release, and methods of use of such combinations and compositions, are provided.

As used herein, the term “renin inhibitor” means an inhibitor of enzymatic activity of renin. Suitable renin inhibitors include without limitation aliskiren, ciprokiren, ditekiren, enalkiren, remikiren, terlakiren and zankiren. Derivatives of these compounds, including salts, esters, prodrugs, metabolites, enantiomers, racemates and tautomers thereof having renin inhibitory properties are also suitable for use in the present invention, as are combinations of renin inhibitors and/or derivatives thereof. Inhibitors of renin release, and derivatives thereof, can likewise be used in the present combinations, compositions and methods.

The term “subject” refers to a warm-blooded animal, generally a mammal such as, for example, a primate, including a human. In one embodiment the subject is a human, for example a patient having hypertension. Typically but not necessarily, hypertension is diagnosed clinically in such a patient.

Blood pressure in most subjects exhibits a 24-hour cycle wherein both systolic and diastolic blood pressures are typically higher during the day than at night. Accordingly, in some embodiments the darusentan and the inhibitor of renin activity or release are present in the combination or composition, or are used in the method, in absolute and relative amounts effective to provide a beneficial change in a subject's 24-hour pattern of SBP and/or DBP.

The term “absolute and relative amounts” as used herein refers to a first amount of darusentan and a second amount of an inhibitor of renin activity or release, wherein the first amount and second amount together are effective to provide the beneficial change recited, whether or not the first amount or second amount alone would be effective.

The term “24-hour pattern” in relation to a blood pressure parameter such as SBP or DBP refers to a cycle in that parameter that recurs approximately daily, for example reflecting underlying endogenous circadian rhythms and/or blood levels of one or more drugs administered in an antihypertensive regimen. For example, increases, decreases, maxima and minima of blood pressure that typically occur each day or night around the same time or times are aspects of the 24-hour pattern. Further aspects include SBP or DBP measured at a specific time in relation to the timing of administration of an antihypertensive drug, for example darusentan and/or an inhibitor of renin activity or release. Illustratively, SBP or DBP measured shortly before the regular time of administration is referred to as “trough” SBP or DBP, being measured at a time when levels of the drug circulating in the bloodstream are assumed to be at their lowest. Thus, where the drug is administered once daily at around 8 am, the trough SBP or DBP relates to a blood pressure measurement taken shortly before 8 am on any day. Blood pressure measurements can be recorded in a sitting or reclining subject. In one embodiment, however, 24-hour pattern and effects of an antihypertensive regimen thereon are established for an ambulatory subject by ambulatory blood pressure (ABP) monitoring.

Examples of beneficial changes in aspects of the 24-hour blood pressure pattern include without limitation
(a) lowering of 24-hour mean ABP;
(b) lowering of trough sitting SBP;
(c) lowering of trough sitting DBP;
(d) lowering of diurnal maximum ABP;
(e) trend away from a bimodal waveform pattern toward a unimodal or less pronouncedly bimodal pattern consistent with normotensive subjects;
(f) increase in day/night ABP ratio; and
(g) at least about 10% nocturnal dipping of ABP.

The present invention can lower any one or more measures of blood pressure as described herein, including SBP and/or DBP as determined, for example, by sphygmomanometry. According to certain embodiments, as indicated with particularity hereinbelow, one or more particular measures of blood pressure are specified.

A “trough sitting” SBP or DBP is measured in a sitting subject at a time point when serum concentration of a drug or drugs is expected to be at or close to its lowest in a treatment cycle, typically just before administration of a further dose. Illustratively, where the drug or drugs are administered once a day at a particular time, for example around 8 am, trough sitting systolic or diastolic blood pressure can be measured at that time, immediately before the daily administration. It is generally preferred to measure trough sitting SBP or DBP at around the same time of day for each such measurement, to minimize variation due to the natural 24-hour blood pressure cycle.

The course of the 24-hour blood pressure cycle is most conveniently traced by ambulatory blood pressure monitoring.

A “24-hour ambulatory” SBP or DBP is an average of measurements taken repeatedly in the course of a 24-hour period, in an ambulatory subject.

A “maximum diurnal” SBP or DBP is a measure of highest SBP or DBP recorded in a 24-hour period, for example by ABP monitoring, and often reflects the peak of the natural 24-hour blood pressure cycle, typically occurring in the morning, for example between about 5 am and about 11 am. Commonly, a second peak occurs in the evening, for example between about 5 pm and 10 pm. Such a bimodal waveform 24-hour ABP pattern may be especially characteristic of resistant hypertension discussed hereinbelow.

Further, a common feature of resistant hypertension is a nighttime (defined herein as 2200 (10 pm) to 0600 (6 am)) mean systolic ABP that is no lower, or lower by a margin of less than about 10%, than the daytime (defined herein as 0600 to 2200) mean systolic ABP. The parameter herein termed “day/night ABP ratio” expressed as a percentage is calculated from daytime and nighttime mean systolic ABP using the formula

(daytime mean−nighttime mean)/daytime mean×100.

A 24-hour ABP pattern having a day/night ABP ratio of less than about 10% is sometimes referred to as a “non-dipping ABP.”

Any dosage of darusentan which, together with the inhibitor of renin activity or release, provides a beneficial effect without unacceptable adverse side-effects in the subject can be present in the combination or composition, or used according to a method of the invention. While in one embodiment darusentan is administered orally, the invention is not limited to any route of administration of darusentan, so long as the route selected results in effective delivery of the drug to provide a beneficial effect. Thus administration of the darusentan can illustratively be parenteral (e.g., intravenous, intraperitoneal, subcutaneous or intradermal), transdermal, transmucosal (e.g., buccal, sublingual or intranasal), intraocular, intrapulmonary (e.g., by inhalation or insufflation) or rectal. Most conveniently for the majority of subjects, however, the darusentan is administered orally, i.e., per os (p.o.). Any suitable orally deliverable dosage form can be used for the darusentan, including without limitation tablets, capsules (solid- or liquid-filled), powders, granules, syrups and other liquids, etc.

For oral administration, any dose of darusentan that, together with the inhibitor of renin activity or release, is therapeutically effective, up to a maximum that is tolerated by the subject without unacceptable adverse side effects, can be administered. For most subjects, such a dose is likely to be about 1 to about 600 mg/day, for example about 5 to about 450 mg/day or about 10 to about 300 mg/day. Higher or lower doses can be useful in specific circumstances.

The inhibitor of renin activity or release may also be present in the combination or composition, or used according to a method of the invention, in any dosage which, together with the darusentan, provides a beneficial effect without unacceptable adverse side-effects in the subject. Although, in one embodiment, the inhibitor of renin activity or release is orally bioavailable and is formulated for oral administration, the invention is not limited to any route of administration of the inhibitor of renin activity or release, so long as the route selected results in effective delivery of the drug to provide a beneficial effect. Further any suitable orally delivery dosage form may be used as indicated above for darusentan.

The prescribed daily dosage amount of the darusentan and/or the inhibitor of renin activity or release can be administered in any suitable number of individual doses, for example four times, three times, twice or once a day. With a dosage form having appropriate controlled release properties, a lower frequency of administration may be possible, for example once every two days, once a week, etc. Administration can be continued for as long as clinically necessary, or for any desired duration, for example as prescribed by a physician. Thus duration of administration can illustratively be about one week to about one year or longer, and in some situations can be continued for substantially the remaining duration of the life of the subject.

Darusentan is suitable for once a day administration, and, where the inhibitor of renin activity or release is likewise suitable for once a day administration, it is generally most convenient to administer both the darusentan and the inhibitor of renin activity or release once a day at around the same time, for example orally in the dosage amounts desired. Where darusentan and the inhibitor of renin activity or release are administered adjunctively with one or more additional drugs, for example drugs constituting an optionally modified baseline therapy as discussed hereinbelow, such additional drugs can often also be administered around the same time.

The darusentan and the inhibitor of renin activity or release may be formulated separately or together in a single pharmaceutical composition as discussed hereinbelow. Further, the darusentan and the inhibitor of renin activity or release may be administered by the same or different routes of administration, and at the same or different times. In one embodiment, both the darusentan and the inhibitor of renin activity or release are formulated for once-daily oral administration, in separate dosage forms or in a single composition.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, darusentan, the inhibitor or renin activity or release, and optionally at least one additional drug useful in combination or adjunctive therapy with the darusentan and inhibitor of renin activity or release. In another example, the darusentan, the inhibitor of renin activity or release and optionally at least one additional drug are separately packaged and available for sale independently of one another, but are co-marketed or co-promoted for use according to the invention. The separate dosage forms can also be presented to a subject separately and independently, for use according to the invention.

Thus one embodiment of the invention provides a therapeutic combination as described above.

In another embodiment of the invention, a pharmaceutical composition is provided comprising such a combination together with at least one pharmaceutically acceptable excipient. The composition can take any suitable form for the desired route of administration. Where the composition is to be administered orally, any suitable orally deliverable dosage form can be used, including without limitation tablets, capsules (solid- or liquid-filled), powders, granules, syrups and other liquids, etc.

Illustratively, a composition that is solid and orally deliverable, for example in a form of a tablet or capsule, typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function. Further information on excipients can be found in standard reference works such as Handbook of Pharmaceutical Excipients, 3rd ed. (Kibbe, ed. (2000), Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A therapeutic combination or pharmaceutical composition of the invention, comprising darusentan and an inhibitor of renin activity or release, optionally further comprises one or more diuretics and/or one or more additional antihypertensive drugs. Such antihypertensive drugs can illustratively be independently selected from (a) ACE inhibitors and angiotensin II receptor blockers, (b) beta-adrenergic receptor blockers, (c) calcium channel blockers, (d) diuretics, (e) direct vasodilators, (f) alpha-1-adrenergic receptor blockers, (g) central alpha-2-adrenergic receptor agonists and other centrally acting antihypertensive drugs, (h) aldosterone receptor antagonists, (i) peripherally acting antihypertensive drugs, (j) vasopeptidase inhibitors, (k) NEP inhibitors, (l) prostanoids, (m) PDE5 inhibitors, (n) nitrosylated compounds and (o) oral nitrates.

Each of these diuretic and antihypertensive drugs, if included in the combination or composition, is typically administered at an adequate to full dose. One of skill in the art can readily identify a suitable dose for any particular diuretic or antihypertensive drug from publicly available information in printed or electronic form, for example on the internet.

Mention of a particular diuretic or antihypertensive drug herein will be understood, except where the context demands otherwise, to include pharmaceutically acceptable salts, esters, prodrugs, metabolites, racemates and enantiomers of the drug, to the extent that such salts, esters, prodrugs, metabolites, racemates or enantiomers exist and are therapeutically effective.

Examples of drugs useful in a combination or composition of the invention are classified and presented in several lists below. Some drugs are active at more than one target; accordingly certain drugs may appear in more than one list. Use of any listed drug in a combination or composition of the invention is contemplated herein, independently of its mode of action.

A suitable diuretic can illustratively be selected from the following list.

Organomercurials
chlormerodrin
meralluride
mercaptomerin sodium
mercumatilin sodium
mercurous chloride
mersalyl

Purines
pamabrom
protheobromine
theobromine

Steroids
canrenone
eplerenone
oleandrin
spironolactone

Sulfonamide Derivatives
acetazolamide
ambuside
azosemide
bumetanide
butazolamide
chloraminophenamide
clofenamide
clopamide
clorexolone
disulfamide
ethoxzolamide
furosemide
mefruside
methazolamide
piretanide
torsemide
tripamide
xipamide

Thiazides and Analogs
althiazide
bendroflumethiazide
benzthiazide
benzylhydrochlorothiazide
buthiazide
chlorothiazide
chlorthalidone
cyclopenthiazide
cyclothiazide
ethiazide
fenquizone
hydrochlorothiazide
hydroflumethiazide
indapamide
methyclothiazide
metolazone
paraflutizide
polythiazide
quinethazone
teclothiazide
trichlormethiazide

Uracils
aminometradine

Unclassified
amiloride
Biogen BG 9719
chlorazanil
ethacrynic acid
etozolin
isosorbide
Kiowa Hakko KW 3902
mannitol
muzolimine
perhexyline
Sanofi-Aventis SR 121463
ticrynafen
triamterene
urea

In some embodiments, the combination or composition comprises a thiazide or loop diuretic. Thiazide diuretics are generally not preferred where the subject has a complicating condition such as diabetes or chronic kidney disease, and in such situations a loop diuretic can be a better choice.

Particularly suitable thiazide diuretics include chlorothiazide, chlorthalidone, hydrochlorothiazide, indapamide, metolazone, polythiazide and combinations thereof. Particularly suitable loop diuretics include bumetanide, furosemide, torsemide and combinations thereof.

A suitable ACE inhibitor can illustratively be selected from the following list:
alacepril
benazepril
captopril
ceronapril
cilazapril
delapril
enalapril
enalaprilat
eosinopril
fosinopril
imidapril
lisinopril
moexipril
moveltipril
omapatrilat
perindopril
quinapril
ramipril
sampatrilat
spirapril
temocapril
trandolapril

Particularly suitable ACE inhibitors include benazepril, captopril, enalapril, fosinopril, lisinopril, moexipril, perindopril, quinapril, ramipril, trandolapril and combinations thereof.

A suitable angiotensin II receptor blocker can illustratively be selected from the following list:
candesartan
eprosartan
irbesartan
losartan
olmesartan
tasosartan
telmisartan
valsartan

A suitable beta-adrenergic receptor blocker can illustratively be selected from the following list:
AC 623
acebutolol
alprenolol
atenolol
amosulalol
arotinolol
atenolol
befimolol
betaxolol
bevantolol
bisoprolol
bopindolol
bucindolol
bucumolol
bufetolol
bufuralol
bunitrolol
bupranolol
butidrine hydrochloride
butofilolol
carazolol
carteolol
carvedilol
celiprolol
cetamolol
cloranolol
dilevalol
esmolol
indenolol
labetalol
landiolol
levobunolol
mepindolol
metipranolol
metoprolol
moprolol
nadolol
nadoxolol
nebivolol
nifenalol
nipradilol
oxprenolol
penbutolol
pindolol
practolol
pronethalol
propranolol
sotalol
sulfinalol
talinolol
tertatolol
tilisolol
timolol
toliprolol
xibenolol

Particularly suitable beta-adrenergic receptor blockers include acebutolol, atenolol, betaxolol, bisoprolol, carvedilol, labetalol, metoprolol, nadolol, penbutolol, pindolol, propranolol, timolol and combinations thereof.

A suitable calcium channel blocker can illustratively be selected from the following list:

Arvklalkylamines
bepridil
clentiazem
diltiazem
fendiline
gallopamil
mibefradil
prenylamine
semotiadil
terodiline
verapamil

Dihydropyridine Derivatives
amlodipine
aranidipine
barnidipine
benidipine
cilnidipine
efonidipine
elgodipine
felodipine
isradipine
lacidipine
lercanidipine
manidipine
nicardipine
nifedipine
nilvadipine
nimodipine
nisoldipine
nitrendipine
NZ 105

Piperazine Derivatives
cinnarizine
dotarizine
flunarizine
lidoflazine
lomerizine

Unclassified
bencyclane
etafenone
fantofarone
monatepil
perhexyline

Particularly suitable calcium channel blockers include amlodipine, diltiazem, felodipine, isradipine, nicardipine, nifedipine, nisoldipine, verapamil and combinations thereof.

A suitable direct vasodilator can illustratively be selected from the following list:
amotriphene
benfurodil hemisuccinate
benziodarone
chloracizine
chromonar
clobenfurol
clonitrate
cloricromen
dilazep
droprenilamine
efloxate
erytlirityl tetranitrate
etafenone
fendiline
hexestrol bis(β-diethylaminoethyl ether)
hexobendine
hydralazine
isosorbide dinitrate
isosorbide mononitrate
itramin tosylate
khellin
lidoflazine
mannitol hexanitrate
minoxidil
nitroglycerin
pentaerythritol tetranitrate
pentrinitrol
perhexyline
pimefyline
prenylamine
propatyl nitrate
trapidil
tricromyl
trimetazidine
trolnitrate phosphate
visnadine

Particularly suitable direct vasodilators include hydralazine, minoxidil and combinations thereof.

A suitable alpha-1-adrenergic receptor blocker can illustratively be selected from the following list:
amosulalol
arotinolol
carvedilol
dapiprazole
doxazosin
ergoloid mesylates
fenspiride
idazoxan
indoramin
labetalol
methyldopa
monatepil
naftopidil
nicergoline
prazosin
tamsulosin
terazosin
tolazoline
trimazosin
yohimbine

Particularly suitable alpha-1-adrenergic receptor blockers include carvedilol, doxazosin, labetalol, prazosin, terazosin and combinations thereof. It is noted that, of these, carvedilol and labetalol also function as beta-adrenergic receptor blockers.

A suitable central alpha-2-adrenergic receptor agonist or other centrally acting antihypertensive drug can illustratively be selected from the following list:
clonidine
guanabenz
guanadrel
guanfacine
methyldopa
moxonidine
reserpine

A suitable aldosterone receptor antagonist can illustratively be selected from the following list:
canrenone
eplerenone
spironolactone

A suitable peripherally acting antihypertensive drug can illustratively be selected from the following list:
guanadrel
guanethidine

Illustrative vasopeptidase inhibitors include:
fasidotril
omapatrilat
sampatrilat

Illustrative NEP inhibitors, some of which are also ACE inhibitors, include:
candoxatril
CGS 26582
MDL 100173
omapatrilat
phosphoramidon
sinorphan
thiorphan
Z13752A

Illustrative prostanoids include:
beraprost
cicaprost
epoprostenol
iloprost
PGE₁
PGI₂ (prostacyclin)
NS-304
treprostinil

Illustrative PDE5 inhibitors include:
sildenafil
tadalafil
vardenafil

Other drugs that can be useful in a combination or composition of the invention can illustratively be selected from the following unclassified list:
ajmaline
alfuzosin
Alteon ALT 711
γ-aminobutyric acid
atrial natriuretic peptide
azelnidipine
bethanidine
bietaserpine
bosentan
budralazine
bufeniode
bunazosin
cadralazine
carmoxirole
CD 3400
chlorisondamine chloride
cicletanine
ciclosidomine
clevidipine
debrisoquin
denitronipradilol
desacetylalacepril
deserpidine
diazoxide
dihydralazine
endralazine
fenoldopam
flosequinan
guanidine, N-cyano-N′-4-pyridinyl-N″-(1,2,2-trimethylpropyl)-, monohydrate
guanoxabenz
guanoxan
hexamethonium
ketanserin
LBI 45
levcromakalim
lofexidine
magnesiocard
mebutamate
mecamylamine
normopresil
2-oxazolamine, N-(dicyclopropylmethyl)-4,5-dihydro-, (2E)-2-butenedioate
pargyline
pempidine
pentamethonium bromide
pentolinium tartrate
pheniprazine
phentolamine
pildralazine
pinacidil
piperoxan
protoveratrines
3,5-pyridinedicarboxylic acid, 1,4-dihydro-2,6-dimethyl-4-(3-nitrophenyl)-, methyl 1-(phenylmethyl)-3-pyrrolidinyl ester
raubasine
rescimetol
rescinnamine
rilmenidine
saralasin
sodium nitroprusside
syrosingopine
Takeda TAK 536
TBC 3711
tetrahydrolipstatin
1,4-thiazepine-4(5H)-acetic acid, 6-[[1-(ethoxycarbonyl)-3-phenylpropyl]-amino]tetrahydro-5-oxo-2-(2-thienyl)
tiamenidine
todralazine
tolonidine
trimethaphan camsylate
tyrosinase
urapidil
zofenopril

In another embodiment, a method is provided for treating a hypertensive disorder in a subject. The method comprises administering to the subject in combination therapy darusentan and an inhibitor of renin activity or release, in absolute and relative amounts effective to provide a beneficial change in the subject's 24-hour pattern of systolic and/or diastolic blood pressure.

Examples of hypertensive disorders that can be treated by the method of this embodiment include conditions marked by systolic hypertension, diastolic hypertension or both, including isolated systolic hypertension and hypertension in the elderly; such conditions can be primary (essential hypertension) or secondary to other conditions including obesity, diabetes, renal disorders (e.g., chronic renal failure, renovascular disease, diabetic nephropathy, etc.), adrenal disorders (e.g., adrenocortical and mineralocorticoid hypertension, pheochromocytoma, primary aldosteronism, Cushing's syndrome, etc.), insulin resistance, salt-sensitivity, polycystic ovary syndrome, sleep apnea, preeclampsia, thyroid and parathyroid diseases, and transplantation. Whether primary or secondary, such hypertension can be, as described above, resistant to baseline antihypertensive therapies, including resistant hypertension as clinically defined or diagnosed. Hypertensive disorders also include pulmonary arterial hypertension, which likewise can be primary or secondary to various conditions including diseases of the scleroderma spectrum (e.g., mixed connective tissue disease, Raynaud's disease, CREST syndrome, systemic sclerosis, or overlap syndrome); rheumatoid arthritis; chronic hepatitis; systemic lupus erythematosus; anorexigen use; human immunodeficiency virus (HIV) infection; chronic hypoxemia resulting from conditions such as chronic bronchitis, emphysema, sleep apnea, interstitial lung disease, or pulmonary fibrosis; thromboembolic diseases such as in situ thrombosis, tumors, or sickle cell disease; volume and pressure overloads induced primarily from disorders of the left heart (for example, chronic heart failure, septal defects, mitral valve disease, and left atrial myxoma); and disorders directly affecting the pulmonary vasculature such as schistosomiasis, sarcoidosis and pulmonary capillary hemangiomatosis.

The present method can be particularly beneficial where the subject having the hypertensive disorder has a compelling or complicating condition such as diabetes, chronic kidney disease or both.

In yet another embodiment, a method is provided for lowering blood pressure in a subject exhibiting resistance to a baseline antihypertensive therapy with one or more drugs. The method comprises administering to the subject in combination therapy darusentan and an inhibitor of renin activity or release.

A “baseline antihypertensive therapy” herein means a therapeutic regimen comprising administration of one or more drugs, not including darusentan, with an objective (which can be the primary objective or a secondary objective of the regimen) of lowering blood pressure in a hypertensive subject. Each drug according to the regimen is administered at least at a dose considered by an attending physician to be adequate for treatment of hypertension, taking into account the particular subject's medical condition and tolerance for the drug without unacceptable adverse side-effects. An “adequate” dose as prescribed by the physician can be less than or equal to a full dose of the drug. A “full” dose is the lowest of (a) the highest dose of the drug labeled for a hypertension indication; (b) the highest usual dose of the drug prescribed according to JNC 7, BHD-IV, ESH/ESC or WHO/ISH guidelines; or (c) the highest tolerated dose of the drug in the particular subject.

A baseline antihypertensive therapy illustratively comprises administering one or more diuretics and/or one or more antihypertensive drugs selected from (a) angiotensin converting enzyme (ACE) inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) peripherally acting antihypertensive drugs, (i) vasopeptidase inhibitors, (j) neutral endopeptidase (NEP) inhibitors, (k) prostanoids, (l) phosphodiesterase type 5 (PDE5) inhibitors, (m) nitrosylated compounds, (n) oral nitrates and (o) inhibitors of renin activity or release; more particularly selected from (a) angiotensin converting enzyme inhibitors and angiotensin II receptor blockers, (b) beta-adrenergic receptor blockers, and (c) calcium channel blockers. Optionally drugs of still further classes can be included in the baseline therapy, for example to address secondary conditions occurring in a hypertensive subject or side-effects of one or more of the diuretic or antihypertensive drugs.

A subject who is “resistant” to a baseline antihypertensive therapy is one in whom hypertension is failing to respond adequately or at all to the baseline therapy. Typically, the subject receiving the baseline therapy is failing to reach an established blood pressure goal, as set forth for U.S. subjects, for example, in JNC 7 or comparable standards in other countries (e.g., BHD-IV, ESH/ESC or WHO/ISH guidelines). Illustratively, the JNC 7 goal for SBP is <140 mmHg and for DBP<90 mmHg, or for a subject having a complicating condition such as diabetes and/or chronic kidney disease, <130 mmHg SBP and <80 mmHg DBP.

The method of the present invention is especially beneficial where the subject has resistant hypertension. By definition herein, such a subject exhibits resistance at least to adequate doses of an appropriate three-drug antihypertensive regimen that includes a diuretic. Typically resistant hypertension is diagnosed clinically. In one embodiment, the subject having resistant hypertension exhibits resistance to a baseline antihypertensive therapy that comprises at least the following:
(l) one or more diuretics; and
(2) two or more antihypertensive drugs, selected from at least two of the following classes:
   (a) ACE inhibitors and angiotensin II receptor blockers;
   (b) beta-adrenergic receptor blockers; and
   (c) calcium channel blockers.

In some cases, the subject is resistant to an even more comprehensive baseline therapy, further comprising, for example, one or more direct vasodilators, alpha-1-adrenergic blockers, central alpha-2-adrenergic agonists or other centrally acting antihypertensive drugs, aldosterone receptor antagonists, vasopeptidase inhibitors, NEP inhibitors, prostanoids, PDE5 inhibitors, nitrosylated compounds, oral nitrates or inhibitors of renin activity or release.

Subjects resistant to a baseline antihypertensive therapy, especially such a therapy involving a plurality of drugs, clearly represent a very challenging population for treatment. Typically in such subjects, increasing dosages of the baseline therapy are not an option because of resulting adverse side effects; furthermore this approach is often ineffective in providing a desired lowering of blood pressure. Accordingly, where the baseline therapy does not already include an inhibitor of renin activity or release, the darusentan and the inhibitor of renin activity or release can be administered adjunctively with the baseline therapy, optionally modified by dose reduction, or even elimination, of at least one of the drugs in the baseline therapy.

Particularly when used at a full dose, many baseline antihypertensive therapy drugs can have undesirable, in some cases clinically unacceptable or even dangerous, adverse side effects.

For example, especially at full doses, potassium-sparing diuretic drugs can be associated with increased risk of hyperkalemia and related disorders. Overuse of loop diuretics can cause depletion of sodium resulting in hyponatremia and/or extracellular fluid volume depletion associated with hypotension, reduced GRF, circulatory collapse, and thromboembolic episodes. Further, loop diuretics can cause ototoxicity that results in tinnitus, hearing impairment, deafness and/or vertigo. Thiazide diuretics, similarly to loop diuretics, can have adverse effects related to abnormalities of fluid and electrolyte balance. Such adverse events include extracellular volume depletion, hypotension, hypokalemia, hyponatremia, hypochloremia, metabolic alkalosis, hypomagnesemia, hypercalcemia and hyperuricemia. Thiazide diuretics can also decrease glucose tolerance, and increase plasma levels of LDL (low density lipoprotein) cholesterol, total cholesterol, and total triglycerides.

Angiotensin converting enzyme (ACE) inhibitors are associated with cough and increased risk of angioedema. Beta-adrenergic receptor blockers are associated with increased risk of bronchospasm, bradycardia, heart block, excess negative inotropic effect, peripheral arterial insufficiency and sometimes male impotence. Calcium channel blockers are associated with increased risk of lower limb edema. Further information on adverse events associated with antihypertensive drugs can be found, for example, in standard reference works such as Goodman & Gilman's The Pharmaceutical Basis of Therapeutics, 13th ed. (Brunton et al., eds. (2006), New York: McGraw Hill).

In situations such as those outlined immediately above, optional modification of the baseline therapy by dose reduction or elimination of a baseline therapy drug permitted by use of darusentan and a inhibitor or renin activity or release can result in a reduced risk or incidence of adverse events by comparison with the baseline therapy alone without such dose reduction or elimination.

“Adjunctive” administration of the darusentan and the inhibitor of renin activity or release herein means that the darusentan and the inhibitor of renin activity or release is administered concomitantly with one or more additional drugs, in the present instance one or more drugs constituting an optionally modified baseline therapy. For example, darusentan and the inhibitor or renin activity or release can optionally be administered adjunctively with an adequate to full dose of one or more of the drugs in the baseline therapy, while the other one or more drugs in the baseline therapy are administered at reduced dose or eliminated.

In one aspect of the present embodiment, the dose and frequency of the darusentan and the inhibitor of renin activity or release administration is effective, for example in combination with the baseline therapy, to provide a reduction of at least about 3 mmHg in trough sitting SBP and/or DBP, 24-hour ambulatory SBP and/or DBP, and/or maximum diurnal SBP and/or DBP.

In a particular aspect, the subject has resistant systolic hypertension, and the dose and frequency of administration of the darusentan and the inhibitor of renin activity or release is effective, for example in combination with the optionally modified baseline therapy, to provide a reduction of at least about 3 mmHg in one or more of trough sitting, 24-hour ambulatory and maximum diurnal SBP.

In a further particular aspect, the at least about 3 mmHg reduction is observed in trough sitting SBP, and at least comparable reductions can be, but are not necessarily, observable in 24-hour ambulatory and/or maximum diurnal SBP. In some cases the method is effective to provide a greater reduction in trough sitting SBP, for example at least about 5 mmHg, at least about 7 mmHg or at least about 10 mmHg.

The present method can increase the likelihood of a subject achieving SBP goal, for example a JNC 7, BHD-IV, ESH/ESC or WHO/ISH goal for SBP. Thus in a still further particular aspect, a JNC 7 goal for SBP is achieved, for example a trough sitting or 24-hour ambulatory SBP of <140 mmHg or, in the case of a subject with diabetes or chronic kidney disease, <130 mmHg.

In another particular aspect, the subject has resistant diastolic hypertension, and the dose and frequency of the administration of darusentan and the inhibitor of renin activity or release is effective, for example in combination with the optionally modified baseline therapy, to provide a reduction of at least about 3 mmHg in one or more of trough sitting, 24-hour ambulatory and maximum diurnal DBP.

In a further particular aspect, the at least about 3 mmHg reduction is observed in trough sitting DBP, and at least comparable reductions can be, but are not necessarily, observable in 24-hour ambulatory and/or maximum diurnal DBP. In some cases the method is effective to provide a greater reduction in trough sitting DBP, for example at least about 5 mmHg, at least about 7 mmHg or at least about 10 mmHg.

The present method can increase the likelihood of a subject achieving DBP goal, for example a JNC 7, BHD-UV, ESH/ESC or WHO/ISH goal for DBP. In a particular embodiment, a JNC 7 goal for DBP is achieved, for example a trough sitting or 24-hour ambulatory DBP of <90 mmHg or, in the case of a subject with diabetes or chronic kidney disease, <80 mmHg.

In yet another embodiment, the present method is effective to provide a beneficial change in the subject's 24-hour pattern of SBP and/or DBP. Examples of the kinds of beneficial changes are listed hereinabove.

Because of the particular criticality of controlling blood pressure in subjects with complicating conditions such as diabetes and/or chronic kidney disease, and the greater difficulty of lowering blood pressure to the lower levels consistent with good management of these conditions, the method of the present embodiment can be especially beneficial for such subjects.

In a particular aspect of the present embodiment, the subject has a compelling or complicating condition such as diabetes, chronic kidney disease or both.

In a still further embodiment, a method is provided for providing a beneficial effect on renal and/or cardiovascular function in a subject having resistant hypertension. The method comprises administering to the subject in combination therapy darusentan and an inhibitor of renin activity or release. “Providing a beneficial effect” in the present context includes enhancing, maintaining or moderating a decline in renal or cardiovascular function, and also includes preventing one or more cardiovascular adverse events. Optionally, the darusentan and the inhibitor of renin activity or release can be administered adjunctively, or in combination, with one or more additional drugs as described herein.

In one aspect of this embodiment, a method is provided for preventing one or more cardiovascular events in a subject having resistant hypertension. The method comprises administering to the subject in combination therapy darusentan and an inhibitor or renin activity or release.

Examples of cardiovascular adverse effects include without limitation acute coronary syndrome (including unstable angina and non-Q wave infarction), myocardial infarction, heart failure, systolic heart failure, diastolic heart failure (also known as diastolic dysfunction), stroke, occlusive stroke, hemorrhagic stroke and combinations thereof. “Preventing” in the present context includes reducing risk, incidence and/or severity of a subsequent cardiovascular adverse effect. Optionally, the darusentan and the inhibitor of renin activity or release can be administered adjunctively, or in combination, with one or more additional drugs as described herein.

In another aspect of the present embodiment, a method for providing a beneficial effect on renal function in a subject having resistant hypertension comprises administering to the subject in combination therapy darusentan and an inhibitor of renin activity or release. A beneficial effect on renal function can be observed, for example, by monitoring one or more blood and/or urinary biomarkers. Examples of such biomarkers include without limitation serum creatinine, serum insulin, serum glutamic acid decarboxylase (GAD), serum protein tyrosine phosphatase-like molecule IA2, blood urea nitrogen, urinary protein, urinary albumin, microalbuminuria, urinary β2-microglobulin, urinary N-acetyl-β-glucosaminidase, urinary retinol binding protein, urinary sodium, glomerular filtration rate, urinary albumin to creatinine ratio, urine volume, and combinations thereof.

Illustratively, the darusentan and the inhibitor of renin activity or release can be administered in absolute and relative amounts effective to lower urinary albumin to creatinine ratio. This can be especially beneficial where the baseline urinary albumin to creatinine ratio is greater than about 30 mg/g or where baseline 24-hour urinary albumin is greater than about 30 mg/day.

In a still further embodiment, darusentan and an inhibitor of renin activity or release are administered as adjunctive therapy for treatment of a patient who is not at goal blood pressure despite adherence to an appropriate antihypertensive drug regimen comprising three or more antihypertensive drugs, including a diuretic. “Goal blood pressure” is as set forth in JNC 7, BHD-IV, ESH/ESC or WHO/ISH guidelines, and is illustratively <140 mmHg SBP and <90 mmHg DBP, or, for a patient having a complicating condition such as diabetes and/or chronic kidney disease, <130 mmHg SBP and <80 mmHg DBP. An “appropriate” antihypertensive drug regimen is one that is normally safe and effective for treatment of at least moderate hypertension, except where resistance to such a regimen is exhibited.

Variants and illustrative modalities of each of the methods described herein, for example suitable inhibitors of renin activity or release, beneficial changes obtained, routes of administration, dosages, formulations, frequency and duration of administration for darusentan and the inhibitor of renin activity or release, and optional additional diuretics and/or additional antihypertensive drugs are as described hereinabove for therapeutic combinations and pharmaceutical compositions of the invention. Thus any combination or composition embraced by the above description may be found suitable for use according to the present methods.

All patents and publications cited herein are incorporated by reference into this application in their entirety.

The words “comprise”, “comprises”, and “comprising” are to be interpreted inclusively rather than exclusively.

What is claimed is:

1. A method for treating a hypertensive disorder in a subject, the method comprising administering to the subject in combination therapy darusentan and an inhibitor of renin activity or release, in absolute and relative amounts effective to provide a beneficial change in the subject's 24-hour pattern of systolic and/or diastolic blood pressure.

2. The method of claim 1, wherein the hypertensive disorder is selected from the group consisting of systolic hypertension; diastolic hypertension; isolated systolic hypertension; hypertension in the elderly; essential hypertension; hypertension secondary to obesity, diabetes, renal disorders, adrenal disorders, Cushing's syndrome, insulin resistance, salt sensitivity, polycystic ovary syndrome, sleep apnea, preeclampsia, thyroid and parathyroid diseases and/or transplantation; and pulmonary arterial hypertension.

3. The method of claim 1, wherein the beneficial change comprises at least one of
(a) lowering of 24-hour mean ambulatory blood pressure;
(b) lowering of trough sitting systolic blood pressure;
(c) lowering of trough sitting diastolic blood pressure;
(d) lowering of diurnal maximum ambulatory blood pressure;
(e) a trend away from a bimodal waveform pattern towards a unimodal or less pronouncedly bimodal pattern consistent with normotensive subjects;
(f) an increase in day/night ambulatory blood pressure ratio; and
(g) at least about 10% nocturnal dipping of ambulatory blood pressure.

4. The method of claim 1, wherein the beneficial change is evident from ambulatory blood pressure monitoring.

5. The method of claim 1, wherein the darusentan is administered at a dose of about 1 to about 600 mg/day.

6. The method of claim 1, wherein the darusentan is administered at a dose of about 10 to about 300 mg/day.

7. The method of claim 1, wherein the inhibitor of renin activity or release is a renin inhibitor.

8. The method of claim 1, wherein the renin inhibitor is selected from the group consisting of aliskiren, ciprokiren, ditekiren, enalkiren, remikiren, terlakiren, zankiren, and salts, esters, prodrugs, metabolites, enantiomers, racemates and tautomers thereof, and combinations thereof.

9. The method of claim 1, wherein at least the darusentan is administered orally.

10. The method of claim 9, wherein the darusentan is orally administered once daily.

11. The method of claim 9, wherein the inhibitor of renin activity or release is orally bioavailable and is administered orally.

12. The method of claim 11, wherein the darusentan and the inhibitor of renin activity or release are each administered orally once daily.

13. The method of claim 12, wherein the darusentan and the inhibitor of renin activity or release are administered together in a single pharmaceutical composition.

14. The method of claim 1, further comprising administering to the subject one or more additional antihypertensive drugs selected from the group consisting of (a) ACE inhibitors and angiotensin II receptor blockers, (b) beta-adrenergic receptor blockers, (c) calcium channel blockers, (d) diuretics, (e) direct vasodilators, (f) alpha-1-adrenergic receptor blockers, (g) central alpha-2-adrenergic receptor agonists and other centrally acting antihypertensive drugs, (h) aldosterone receptor antagonists, (i) vasopeptidase inhibitors, (j) NEP inhibitors, (k) prostanoids, (l) PDE5 inhibitors, (m) nitrosylated compounds and (n) oral nitrates.

15. The method of claim 1, wherein the subject has diabetes, chronic kidney disease or both.

16. A method for lowering blood pressure in a subject exhibiting resistance to a baseline antihypertensive therapy with one or more drugs, the method comprising administering to the subject in combination therapy darusentan and an inhibitor of renin activity or release.

17. The method of claim 16, wherein the baseline therapy comprises administration of one or more diuretics and/or one or more antihypertensive drugs selected from the group consisting of (a) ACE inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) NEP inhibitors, (j) prostanoids, (k) PDE5 inhibitors, (l) nitrosylated compounds, (m) oral nitrates and (n) inhibitors of renin activity or release.

18. The method of claim 16, wherein the subject has resistant hypertension, and the baseline therapy comprises administration of at least one diuretic and at least two antihypertensive drugs selected from at least two of (a) ACE inhibitors and angiotensin II receptor blockers, (b) beta-adrenergic receptor blockers and (c) calcium channel blockers.

19. The method of claim 18, wherein the darusentan and the inhibitor of renin activity or release are administered adjunctively with the baseline therapy, optionally modified by dose reduction or elimination of one or more of the baseline therapy drugs.

20. The method of claim 19, wherein the subject has resistant systolic hypertension, and the darusentan and the inhibitor of renin activity or release are administered in absolute and relative amounts effective, in combination with the optionally modified baseline therapy, to provide a reduction of at least about 3 mmHg in one or more systolic blood pressure parameters selected from trough sitting, 24-hour ambulatory and maximum diurnal systolic blood pressures.

21. The method of claim 20, wherein a JNC 7, BHD-IV, ESH/ESC or WHO/ISH goal for systolic blood pressure is achieved.

22. The method of claim 19, wherein the subject has resistant diastolic hypertension, and the darusentan and the inhibitor of renin activity or release are administered in absolute and relative amounts effective, in combination with the optionally modified baseline therapy, to provide a reduction of at least about 3 mmHg in one or more diastolic blood pressure parameters selected from trough sitting, 24-hour ambulatory and maximum diurnal diastolic blood pressures.

23. The method of claim 22, wherein a JNC 7, BHD-IV, ESH/ESC or WHO/ISH goal for diastolic blood pressure is achieved.

24. The method of claim 16, wherein a beneficial change in the subject's 24-hour pattern of systolic and/or diastolic blood pressure is obtained.

25. The method of claim 24, wherein the beneficial change comprises at least one of
(a) lowering of 24-hour mean ambulatory blood pressure;
(b) lowering of trough sitting systolic blood pressure;
(c) lowering of trough sitting diastolic blood pressure;
(d) lowering of diurnal maximum ambulatory blood pressure;
(e) a trend away from a bimodal waveform pattern towards a unimodal or less pronouncedly bimodal pattern consistent with normotensive subjects;
(f) an increase in day/night ambulatory blood pressure ratio; and
(g) at least about 10% nocturnal dipping of ambulatory blood pressure.

26. The method of claim 24, wherein the beneficial change is evident from ambulatory blood pressure monitoring.

27. The method of claim 16, wherein the darusentan is administered at a dose of about 1 to about 600 mg/day.

28. The method of claim 16, wherein the darusentan is administered at a dose of about 10 to about 300 mg/day.

29. The method of claim 16, wherein the inhibitor of renin activity or release is a renin inhibitor.

30. The method of claim 29, wherein the renin inhibitor is selected from the group consisting of aliskiren, ciprokiren, ditekiren, enalkiren, remikiren, terlakiren, zankiren, and salts, esters, prodrugs, metabolites, enantiomers, racemates and tautomers thereof, and combinations thereof.

31. The method of claim 16, wherein the subject has diabetes, chronic kidney disease or both.

32. A method for providing a beneficial effect on renal and/or cardiovascular function in a subject having resistant hypertension, the method comprising administering to the subject in combination therapy darusentan and an inhibitor of renin activity or release.

33. The method of claim 32, wherein the beneficial effect comprises preventing one or more cardiovascular adverse events in the subject.

34. The method of claim 32, wherein the one or more cardiovascular adverse events are selected from the group consisting of acute coronary syndrome, myocardial infarction, heart failure, systolic heart failure, diastolic heart failure, stroke, occlusive stroke, hemorrhagic stroke and combinations thereof.

35. The method of claim 32, wherein the beneficial effect is on renal function and is observable by monitoring one or more blood and/or urinary biomarkers.

36. The method of claim 35, wherein the one or more biomarkers are selected from the group consisting of serum creatinine, serum insulin, serum GAD, serum IA2, blood urea nitrogen, urinary protein, urinary albumin, microalbuminuria, urinary β2-microglobulin, urinary N-acetyl-β-glucosaminidase, urinary retinol binding protein, urinary sodium, glomerular filtration rate, urinary albumin to creatinine ratio, urine volume and combinations thereof.

37. The method of claim 35, wherein the darusentan and the inhibitor of renin activity or release are administered in absolute and relative amounts effective to lower urinary albumin to creatinine ratio.

38. The method of claim 37, wherein the subject exhibits, prior to administration of the darusentan and the inhibitor of renin activity or release, a baseline urinary albumin to creatinine ratio greater than about 30 mg/g.

39. The method of claim 37, wherein the subject exhibits, prior to administration of the darusentan and the inhibitor of renin activity or release, a baseline 24-hour urinary albumin greater than about 30 mg/day.

40. The method of claim 32, wherein the darusentan is administered at a dose of about 1 to about 600 mg/day.

41. The method of claim 32, wherein the darusentan is administered at a dose of about 10 to about 300 mg/day.

42. The method of claim 32, wherein the inhibitor of renin activity or release is a renin inhibitor.

43. The method of claim 42, wherein the renin inhibitor is selected from the group consisting of aliskiren, ciprokiren, ditekiren, enalkiren, remikiren, terlakiren, zankiren, and salts, esters, prodrugs, metabolites, enantiomers, racemates and tautomers thereof, and combinations thereof.

44. A therapeutic combination comprising darusentan and an inhibitor of renin activity or release in absolute and relative amounts effective to provide a beneficial change in a subject's 24-hour pattern of systolic and/or diastolic blood pressure, wherein the darusentan and the inhibitor of renin activity or release are each formulated for once-daily oral administration.

45. The combination of claim 44, wherein the beneficial change comprises at least one of
(a) lowering of 24-hour mean ambulatory blood pressure;
(b) lowering of trough sitting systolic blood pressure;
(c) lowering of trough sitting diastolic blood pressure;
(d) lowering of diurnal maximum ambulatory blood pressure;
(e) a trend away from a bimodal waveform pattern towards a unimodal or less pronouncedly bimodal pattern consistent with normotensive subjects;
(f) an increase in day/night ambulatory blood pressure ratio; and
(g) at least about 10% nocturnal dipping of ambulatory blood pressure.

46. The combination of claim 44, comprising darusentan in an amount providing a dose of about 1 to about 600 mg/day.

47. The combination of claim 44, comprising darusentan in an amount providing a dose of about 10 to about 300 mg/day.

48. The combination of claim 44, wherein the inhibitor of renin activity or release is a renin inhibitor.

49. The combination of claim 48, wherein the renin inhibitor is selected from the group consisting of aliskiren, ciprokiren, ditekiren, enalkiren, remikiren, terlakiren, zankiren, and salts, esters, prodrugs, metabolites, enantiomers, racemates and tautomers thereof, and combinations thereof.

50. The combination of claim 44, wherein the darusentan and the inhibitor of renin activity or release are separately formulated for administration by the same or different routes at the same or different times.

51. The combination of claim 44, further comprising one or more additional antihypertensive drugs selected from the group consisting of (a) ACE inhibitors and angiotensin II receptor blockers, (b) beta-adrenergic receptor blockers, (c) calcium channel blockers, (d) diuretics, (e) direct vasodilators, (f) alpha-1-adrenergic receptor blockers, (g) central alpha-2-adrenergic receptor agonists and other centrally acting antihypertensive drugs, (h) aldosterone receptor antagonists, (i) vasopeptidase inhibitors, (j) NEP inhibitors, (k) prostanoids, (l) PDE5 inhibitors, (m) nitrosylated compounds and (n) oral nitrates.

52. A pharmaceutical composition comprising darusentan, an inhibitor of renin activity or release, and at least one pharmaceutically acceptable excipient; wherein the darusentan and the inhibitor of renin activity or release are present in absolute and relative amounts effective to provide a beneficial change in a subject's 24-hour pattern of systolic and/or diastolic blood pressure; and wherein the composition is formulated for once-daily oral administration.

53. The composition of claim 52, wherein the beneficial change comprises at least one of
(a) lowering of 24-hour mean ambulatory blood pressure;
(b) lowering of trough sitting systolic blood pressure;
(c) lowering of trough sitting diastolic blood pressure;
(d) lowering of diurnal maximum ambulatory blood pressure;
(e) a trend away from a bimodal waveform pattern towards a unimodal or less pronouncedly bimodal pattern consistent with normotensive subjects;
(f) an increase in day/night ambulatory blood pressure ratio; and
(g) at least about 10% nocturnal dipping of ambulatory blood pressure.

54. The composition of claim 52, comprising darusentan in an amount providing a dose of about 1 to about 600 mg/day.

55. The composition of claim 52, comprising darusentan in an amount providing a dose of about 10 to about 300 mg/day.

56. The composition of claim 52, wherein the inhibitor of renin activity or release is a renin inhibitor.

57. The composition of claim 56, wherein the renin inhibitor is selected from the group consisting of aliskiren, ciprokiren, ditekiren, enalkiren, remikiren, terlakiren, zankiren, and salts, esters, prodrugs, metabolites, enantiomers, racemates and tautomers thereof, and combinations thereof.

58. The composition of claim 52, further comprising one or more additional antihypertensive drugs selected from the group consisting of (a) ACE inhibitors and angiotensin II receptor blockers, (b) beta-adrenergic receptor blockers, (c) calcium channel blockers, (d) diuretics, (e) direct vasodilators, (f) alpha-1-adrenergic receptor blockers, (g) central alpha-2-adrenergic receptor agonists and other centrally acting antihypertensive drugs, (h) aldosterone receptor antagonists, (i) vasopeptidase inhibitors, (j) NEP inhibitors, (k) prostanoids, (l) PDE5 inhibitors, (m) nitrosylated compounds and (n) oral nitrates.
